(19) United States
(12) Patent Application Publication
(10) Pub. No.: US 2017/0299483 A1
(43) Pub. Date: Oct. 19, 2017

(54) DEVICES AND METHODS FOR PREPARING BIOLOGICAL SAMPLES

(71) Applicant: CALIFORNIA INSTITUTE OF TECHNOLOGY, Pasadena, CA (US)

(72) Inventors: Rustem F. Ismagilov, Altadena, CA (US); Erik Jue, Pasadena, CA (US); Nathan Schoepp, Pasadena, CA (US)

(21) Appl. No.: 15/485,431

(22) Filed: Apr. 12, 2017

Related U.S. Application Data
(60) Provisional application No. 62/324,150, filed on Apr. 18, 2016.

(51) Int. Cl. G01N 1/38; B33Y 10/00; C12Q 1/68; B33Y 80/00; B01L 3/00

(52) U.S. Cl. CPC G01N 1/38; B33Y 80/00; B01L 3/502; B33Y 10/00; C12Q 1/6806; B01L 2200/026; B01L 2200/0605; B01L 2400/0478; B01L 2400/06; G01N 2001/382; B01L 2200/12

(57) ABSTRACT

Devices and methods for biological sample preparation are provided herein. Components of such devices include a plurality of plungers that can be actuated for metering and/or mixing one or more agents. Methods of manufacturing such devices using 3D printing are also provided.

### CROSS-REFERENCE TO RELATED APPLICATIONS

This application claims the benefit under 35 USC 119(e) of prior co-pending U.S. Provisional Patent Application No. 62/324,150, filed Apr. 18, 2016, the disclosure of which is hereby incorporated by reference in its entirety.

### STATEMENT REGARDING FEDERALLY SPONSORED RESEARCH OR DEVELOPMENT

This invention was made with government support under Grant No. HR0011-11-2-0006 awarded by DARPA and under Grant No. DGE1144469 awarded by the National Science Foundation. The government has certain rights in the invention.

### INTRODUCTION

Efforts have been taken to optimize sample preparation for assays, including those involving nucleic acid (NA) amplification. As part of such efforts, sample metering has been an area of interest because, for example, a user in limited-resource settings (LRS) or at the point of care (POC) can face challenges in pipetting accurately. Precise metering is especially important in nucleic acid amplification testing (NAAT) testing of particular diseases including sexually transmitted diseases (STDs), such as Chlamydia trachomatis (CT) and Neisseria gonorrhoeae (NG). (12). In 2013, there were 1,401,906 and 333,004 reported cases of CT and NG, respectively, in the United States, with many more cases unreported and undiagnosed. (13). The Centers for Disease Control and Prevention (CDC) estimates 20 million new STD infections per year in the US, accounting for $16 billion in health care costs. (13). The CDC now recommends NAAT for CT/NG diagnosis (14) because these tests are sensitive, accurate and use non-invasive urine samples. Many of these tests need to be done under LRS or POC settings.

### SUMMARY

Devices and methods for biological sample preparation are provided herein. Components of such devices include a plurality of plungers that can be actuated for metering and/or mixing one or more agents. Methods of manufacturing such devices using 3D printing are also provided.

Disclosed embodiments include a biological assay sample preparation device, the device including: a first container and a second container; a first plunger actuable within the first container in a first direction and a second direction opposite the first direction; a second plunger actuable within the second container in the first direction and the second direction; and/or a valve actuable between a first and second configuration. In some versions, the first container is fluidically connected to the second container when the valve is in the second configuration and not fluidically connected to the second container when the valve is in the first configuration, and in some versions, the first plunger and the second plunger are concertedly actuable in the second direction toward the valve when the valve is in the second configuration.

According to various embodiments, the first plunger includes a first locking element and the valve includes a first opening for receiving one or more portion of the first locking element therein in the first configuration. In some versions the valve includes a second opening for receiving one or more portion of the first locking element therein in the second configuration. The second plunger can also include a second locking element preventing the second plunger from actuating in the first direction when the valve is in the first valve configuration. In some versions the valve includes a second opening for receiving one or more portion of the second locking element therein in the second configuration.

The subject devices can also include a third container, wherein the third container is not fluidically connected to the first or second container when the valve is in the first configuration and is fluidically connected to the first and second container when the valve is in the second configuration. The third container can include an outlet.

According to various aspects, the second container includes a preparation solution, such as a lysing agent and/or a buffer solution. A device can also include an inlet conduit, wherein the first container is fluidically connected to the inlet conduit when the valve is in the first configuration and not fluidically connected to the inlet conduit when the valve is in the second configuration.

The devices as disclosed herein can also include a housing containing the first and second containers. In some versions, the valve is slidably coupled to the housing and slides within the housing to actuate between the first and second configuration. Also, in some aspects, the valve includes an inner core and an outer layer surrounding the inner core, wherein the inner core includes a first material and the outer layer includes a second material, e.g., an elastomeric material, different than the first material, e.g., a non-elastomeric material.

In some aspects, the first plunger includes an inner core and an outer layer surrounding the inner core, wherein the inner core includes a first material and the outer layer includes a second material, e.g., an elastomeric material, different than the first material. In some versions of the devices the second plunger includes an inner core and an outer layer surrounding the inner core, wherein the inner core includes a first material and the outer layer includes a second material, e.g., an elastomeric material, different than the first material.

Also, according to various embodiments, the first container defines a first cross-sectional area and the second container defines a second cross-sectional area, and wherein the ratio of the first cross-sectional area to the second cross-sectional area is 1:2 or less, or 2:1 or less, or 1:2 or greater or 2:1 or greater.

Also provided herein are methods according to various embodiments including methods of preparing a biological sample. In some aspects, the methods include: advancing a first plunger of a biological assay sample preparation device within a first container of the device to move a biological sample into the first container; actuating a valve of the device from a first configuration to a second configuration and thereby fluidically connecting the first container with a second container of the device including a preparation solution; and/or advancing the first plunger in the first container and the second plunger in the second container of the device in concerted motion toward the valve. In some versions, advancing the first plunger and/or the second plunger in concerted motion prepares the biological sample by mixing the biological sample and the preparation solution.

According to various aspects of the methods, the first plunger includes a first locking element and the valve includes a first opening for receiving one or more portion of the first locking element therein, and wherein advancing the first plunger within the first container to move the biological sample into the first container includes removing the first locking element from the first opening. In some versions, the second plunger includes a second locking element and wherein advancing the first plunger within the first container to move the biological sample into the first container includes contacting the second locking element with the valve and thereby blocking the second plunger from actuating in the first direction.

In some variations of the methods, the valve includes a second opening for receiving one or more portion of the second locking element therein in the second configuration, and wherein advancing the first plunger and the second plunger in concerted motion includes inserting one or more portion of the second locking element into the second opening. In various embodiments, advancing the first plunger in the first container and the second plunger in the second container of the device in concerted motion includes flowing the biological sample and the preparation solution into a third container of the device. According to various aspects, actuating the valve from a first configuration to a second configuration includes fluidically connecting the first container and the second container of the device with the third container.

Advancing the first plunger in the first container and the second plunger in the second container of the device in concerted motion, in some aspects, further includes flowing the biological sample and the preparation solution out of the third container of the device. In some versions of the methods, the device further includes an inlet conduit and wherein actuating the valve from a first configuration to a second configuration includes fluidically disconnecting the inlet conduit from the first container.

According to various embodiments, the first container includes an inlet, wherein the valve includes an inner core and an outer layer surrounding the inner core, wherein the inner core includes a first material and the outer layer includes a second material different than the first material, and wherein actuating the valve from a first configuration to a second configuration includes fluidically sealing the inlet with the second material. Also, in some aspects advancing the first plunger and the second plunger in concerted motion includes moving the first plunger and the second plunger in a single direction to push the biological sample out of the first container and the preparation solution out of the second container.

In various embodiments, the biological sample includes cells and mixing the biological sample and the preparation solution includes lysing the cells with the preparation solution. According to various aspects, advancing the first plunger and the second plunger in concerted motion propels a first volume of biological sample out of the first container and propels a second volume of preparation solution out of the second container, wherein the ratio of the first volume to the second volume is 1:2 or less or 2:1 or less, or 1:2 or greater or 2:1 or greater.

Also disclosed herein are methods of manufacturing a biological assay sample preparation device. Such methods can include 3-dimensionally (3D) printing device components including: a first container and a second container; a first plunger actuable within the first container in a first direction and a second direction opposite the first direction; a second plunger actuable within the second container in the first direction and the second direction; and/or a valve actuable between a first and second configuration, and/or assembling the components to produce a biological assay sample preparation device.

In various aspects, when assembled, the first container is fluidically connected to the second container when the valve is in the second configuration and not fluidically connected to the second container when the valve is in the first configuration. According to some embodiments, when assembled, the first plunger and the second plunger are concertedly actuable in the second direction toward the valve when the valve is in the second configuration.

In some variations, the first plunger includes a first locking element preventing the first plunger from actuating in the second direction when the valve is in the first valve configuration. In some aspects, the second plunger includes a second locking element preventing the second plunger from actuating in the first direction when the valve is in the first valve configuration. In some embodiments of the devices, the device components include a third container, wherein the third container is not fluidically connected to the first or second container when the valve is in the first configuration and is fluidically connected to the first and second container when the valve is in the second configuration.

According to some aspects, the valve includes an inner core and an outer layer surrounding the inner core, wherein the inner core includes a first material and the outer layer includes a second material different than the first material, and wherein 3-dimensionally printing the valve includes printing the inner core with the first material, such as a non-elastomeric material, and then the outer core with the second material, such as an elastomeric material.

In some versions, the first plunger includes an inner core and an outer layer surrounding the inner core, wherein the inner core includes a first material and the outer layer includes a second material different than the first material, and wherein 3-dimensionally printing the first plunger includes printing the inner core with the first material, such as a non-elastomeric material, and then the outer core with the second material, such as an elastomeric material. In some variations the second plunger includes an inner core and an outer layer surrounding the inner core, wherein the inner core includes a first material and the outer layer includes a second material different than the first material, and wherein 3-dimensionally printing the second plunger includes printing the inner core with the first material, such as a non-elastomeric material, and then the outer core with the second material, such as an elastomeric material.

Also, according to various aspects, the first container defines a first cross-sectional area and the second container defines a second cross-sectional area, and wherein the ratio of the first cross-sectional area to the second cross-sectional area is 1:2 or less, or 2:1 or less, or 1:2 or greater or 2:1 or greater.

### BRIEF DESCRIPTION OF THE DRAWINGS

These and other aspects and features of the present invention will become apparent to those ordinarily skilled in the art upon review of the following description of specific embodiments of the invention in conjunction with the accompanying figures, wherein:

FIG. 1 provides a side-perspective view of a device according to the subject embodiments.

FIGS. 2A-E provide side-perspective views of a device having components in various conformations according to the subject embodiments.

FIGS. 3A-D provide a side-perspective view of a device according to the subject embodiments.

FIGS. 4A-G illustrate embodiments of components of the subject devices such as third containers and/or mixing elements therein.

FIG. 5 provides data relating to the function and biocompatibility of a tested device.

### DETAILED DESCRIPTION

Devices and methods for biological sample preparation are provided herein. Components of such devices include a plurality of plungers that can be actuated for metering and/or mixing one or more agents. Methods of manufacturing such devices using 3D printing are also provided.

Before the present invention is described in greater detail, it is to be understood that this invention is not limited to particular embodiments described, as such can, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges can independently be included in the smaller ranges and are also encompassed within the invention, subject to any specifically excluded limit in the stated range. Where the stated range includes one or both of the limits, ranges excluding either or both of those included limits are also included in the invention.

Certain ranges can be presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided can be different from the actual publication dates which can need to be independently confirmed.

It is noted that, as used herein and in the appended claims, the singular forms “a,” “an,” and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

Additionally, certain embodiments of the disclosed devices and/or associated methods can be represented by drawings which can be included in this application. Embodiments of the devices and their specific spatial characteristics and/or abilities include those shown or substantially shown in the drawings or which are reasonably inferable from the drawings. Such characteristics include, for example, one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal; distal), and/or numbers (e.g., three surfaces; four surfaces), or any combinations thereof. Such spatial characteristics also include, for example, the lack (e.g., specific absence of) one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal), and/or numbers (e.g., three surfaces), or any combinations thereof.

As will be apparent to those of skill in the art upon reading this disclosure, each of the individual embodiments described and illustrated herein has discrete components and features which can be readily separated from or combined with the features of any of the other several embodiments without departing from the scope or spirit of the present invention. Any recited method can be carried out in the order of events recited or in any other order which is logically possible.

In further describing the subject invention, subject devices for use in practicing the subject methods will be discussed in greater detail, followed by a review of associated methods. DEVICES

Devices for biological sample preparation are provided herein. Components of such devices include a plurality of plungers that can be actuated for metering and/or mixing one or more agents to prepare a sample. Such metering and mixing can include isolating one or more specific quantity of one or more substances in particular containers of a device at and/or mixing two or more quantities of such substances such in a container in their entirety to produce an intended mixture.

Embodiments of the subject disclosure include biological assay sample preparation devices. As used herein, a “biological assay” is test on a biological sample that is performed to evaluate one or more characteristics of the sample. A biological sample is a sample containing a quantity of organic material, e.g., one or more organic molecules, such as one or more nucleic acids e.g., DNA and/or RNA or portions thereof, that can be taken from a subject. A biological sample can include one or more of blood, urine, mucus, or other body fluid. Accordingly, biological assay sample preparation devices, according to some embodiments, are devices that prepare a biological sample for analysis with a biological assay. Also, in some aspects a biological sample is a nucleic acid amplification sample, which is a sample including one or more nucleic acids or portions thereof that can be amplified according to the subject embodiments.

Biological samples can be collected from a subject and can include one or more cells, such as tissue cells of the subject. As used herein, the term “tissue” refers to one or more aggregates of cells in a subject (e.g., a living organism, such as a mammal, such as a human) that have a similar function and structure or to a plurality of different types of such aggregates. Tissue can include, for example, organ tissue, muscle tissue (e.g., cardiac muscle; smooth muscle; and/or skeletal muscle), connective tissue, nervous tissue and/or epithelial tissue. A biological sample can also not include one or more cells. In some embodiments, a biological sample can include free DNA, free RNA, viral particles, bacteria cells or cell portions, fungi, prions, spores, or any combination thereof.

A biological sample can be collected from a subject. In certain embodiments, a subject is a “mammal” or a “mammalian” subject, where these terms are used broadly to describe organisms that are within the class mammalia, including the orders carnivore (e.g., dogs and cats), rodentia (e.g., mice, guinea pigs, and rats), and primates (e.g., humans, chimpanzees, and monkeys). In some embodiments, the subject is a human. The term “humans” can include human subjects of both genders and at any stage of development (e.g., fetal, neonates, infant, juvenile, adolescent, and adult), where in certain embodiments the human subject is a juvenile, adolescent or adult. While the devices and methods described herein can be applied in association with a human subject, it is to be understood that the subject devices and methods can also be applied in association with other subjects, that is, on “non-human subjects.”

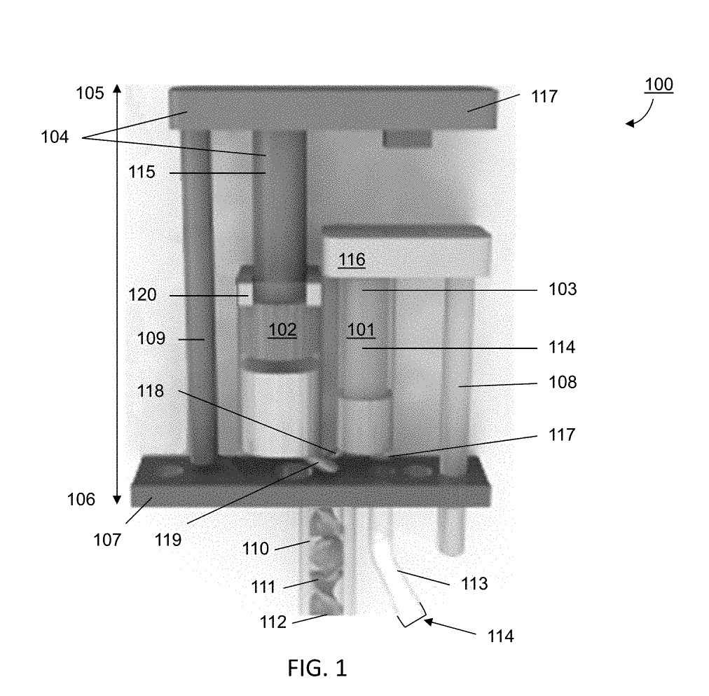

One embodiment of a biological assay sample preparation device for use in practicing the subject methods is provided in FIG. 1. In various embodiments, the biological assay sample preparation device 100 includes a first container 101 and a second container 102. The device also can include a first plunger 103 actuable within the first container 101 in a first direction, e.g., as represented by arrow 105, and a second direction, e.g., as represented by arrow 106, opposite the first direction 105. Embodiments of the device include a second plunger actuable 104 within the second container 102 in the first direction 105 and the second direction 106.

A valve 107 actuable between a first and second configuration also can be included in the device 100. In various aspects, the first container 101 is fluidically connected to the second container 102 when the valve 107 is in the second valve configuration and/or not fluidically connected to the second container 102 when the valve 107 is in the first valve configuration. Also, in some aspects the first plunger 103 and the second plunger 104 are concertedly actuable in the second direction 106 toward the valve 107 when the valve 102 is in the second configuration.

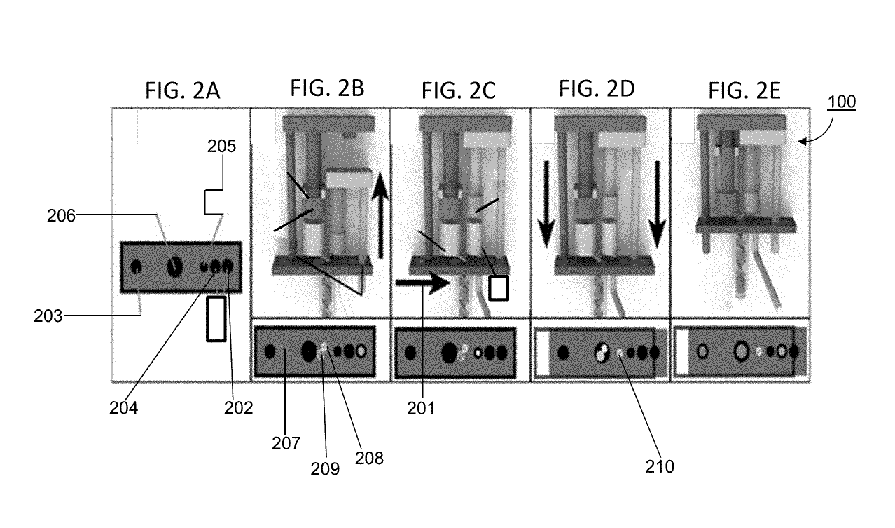

The valve 107 is shown in a first configuration, for example, in FIGS. 2B and 2C. The valve is moved in a direction, such as the direction provided by arrow 201, which can be perpendicular or substantially perpendicular to the first direction, e.g., as represented by arrow 105, and/or the second direction, e.g., as represented by arrow 106 to the second configuration as shown in FIGS. 2D and 2E. As used herein, “substantially” means to a great or significant extent, such as almost fully or almost entirely.

As shown, for example, in FIG. 2A, the valve 107 can include a planar plate, such as a rectangular plate, having a thickness extending from a first surface to a second surface opposite the first surface. The valve 107 can include one or more, e.g., 2, 3, 4, 5, 6, 7, 8, 9 or 10 openings therein. The valve 107 can also be actuable by sliding, e.g., manually or automatically sliding, the valve, such as sliding the valve within a housing, such as a housing including a valve receptacle including one or more valve-receiving grooves in which the valve can slide. Actuating the valve can be performed by exerting force on the valve, such as on an end of the valve, such as by contacting the valve and exerting force in a direction, e.g., 201, thereon.

The openings can include a first opening 202 for receiving therein and/or therethrough a first locking element in the first valve configuration; a second opening 203 for receiving therein and/or therethrough a second locking element in the second valve configuration and/or not in the first valve configuration; a third opening 204 for receiving therein and/or therethrough a first locking element in the second valve configuration; a fourth opening 205 that can be operatively coupleable to a fluid conduit such as an inlet conduit 113 and/or can be configured to allow a fluid, such as a biological sample, to flow therethrough from an inlet; and/or a fifth opening 206 operatively coupleable to the first container 101 and/or the second container 102 and that can be configured to allow a fluid, such as a biological sample to flow therethrough to an outlet. Such openings can extend through the valve from the first surface to the second surface and can be circular, rectangular, square, triangular, or any combination thereof.

By “operatively coupled,” “operatively connected” and “operatively attached” as used herein, is meant connected in a specific way that allows the disclosed devices to operate and/or methods to be carried out effectively in the manner described herein. For example, operatively coupling can include removably coupling or fixedly coupling two or more aspects. Operatively coupling can also include fluidically and/or electrically and/or mateably and/or adhesively coupling two or more components. Also, by “removably coupled,” as used herein, is meant coupled, e.g., physically and/or fluidically and/or electrically coupled, in a manner wherein the two or more coupled components can be un-coupled and then re-coupled repeatedly.

Valves as provided herein can also include one or more e.g., 1, 2, 3, 4, 5, 6, 7, 8, 9 or 10 blocking portions, such as surface portions that contact a portion of a first and/or second plunger, or a portion thereof, e.g., a first and/or second locking element, and thereby prevent the plunger from actuating while, for example, the valve is in a first and/or second configuration. Such blocking portions, can also be portions that contact a portion of a first and/or second and/or third container, such as an inlet and/or an outlet, and thereby prevent fluid from moving into and/or out of the container while, for example, the valve is in a first and/or second configuration.

For example, as is shown in FIGS. 2B-E, a valve can include a first blocking portion that contacts a locking element of a second plunger and thereby prevents the plunger from substantially actuating while, for example, the valve is in a first configuration. A valve can include a second blocking portion 208 that contacts and thereby covers and seals an outlet of a first container and thereby prevents fluid from moving into and/or out of the container through the outlet while, for example, the valve is in a first configuration. A valve can include a third blocking portion 209 that contacts and thereby covers and seals an outlet of a second container and thereby prevents fluid from moving into and/or out of the container through the outlet while, for example, the valve is in a first configuration. A valve can include a fourth blocking portion 210 that contacts and thereby covers and seals an inlet of a first container and thereby prevents fluid from moving into and/or out of the container through the inlet while, for example, the valve is in a second configuration.

In some versions, the subject devices or portions thereof, such as a first plunger 103, can include a first locking element 108. A first locking element can be configured to prevent the first plunger from actuating in the second direction when the valve is in the first valve configuration. Also, the subject devices or portions thereof, such as a second plunger 104, can include a second locking element 109. A second locking element can be configured to prevent the second plunger from actuating in the first direction when the valve is in the first valve configuration.

In some aspects, a first container 101 includes an inlet 117 and/or an outlet 118. Such an inlet 117 can be operably connectable with the inlet conduit 113 as described herein such that fluid can flow from the inlet conduit 113 into the first container via the inlet 117 when, for example, the valve 107 is in a first configuration. Such an inlet 117 can be sealed by the valve 107 when the valve moves from the first configuration to the second configuration. Also, an outlet 118 can be operably connectable with a third container 110 as described herein such that fluid can flow from the outlet 118 into the third container via the outlet 118 when, for example, the valve is in a second configuration. Such an outlet 118 can be sealed by the valve 107 when the valve is in the first configuration.

Also, according to some versions of the subject disclosure, a second container 102 includes an outlet 119. Such an outlet 119 can be operably connectable with the third container 110 as described herein such that fluid can flow from the second container 102 into the third container 110 via the outlet 119 when, for example, the valve 107 is in a second configuration. Such an outlet 119 can be sealed by the valve 107 when the valve is in the first configuration.

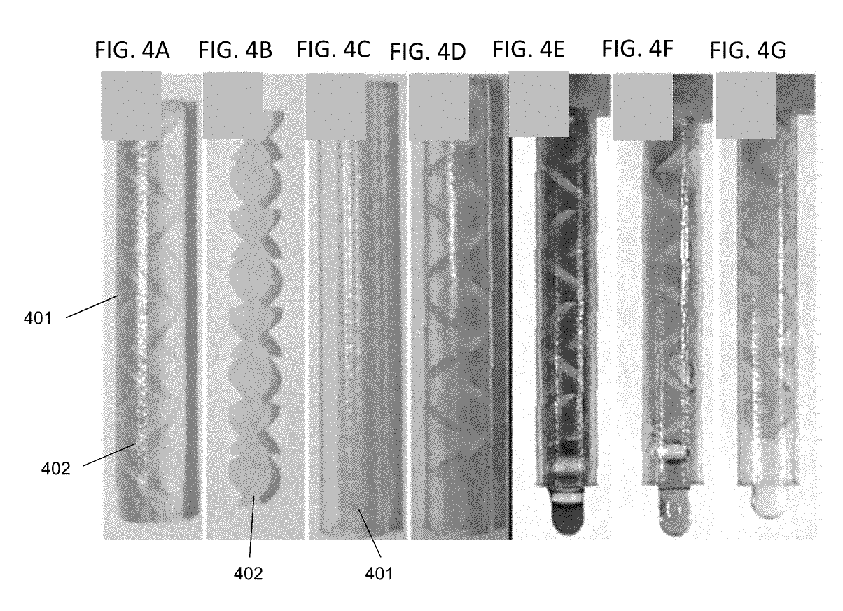

In some embodiments, the device 100 includes a third container 110. In some versions, the third container is not fluidically connected to the first or second container when the valve is in the first configuration and/or is fluidically connected to the first and second container when the valve is in the second configuration. The third container can also include one or more mixing element 111 that can be configured to provide turbulent fluid flow through the container 110 and thus provide for effective mixing of liquids within the container 110. Various embodiments of third containers and/or mixing elements therein are shown, for example in FIGS. 4A-G.

In various embodiments, the third container includes one or more outlet 112. Such an outlet 112 can include one or more opening and can be configured to provide fluid flow therethrough such that fluids, such as a prepared biological sample, can flow out of the device 100. In some versions, the third container 110 is operably connected to the valve 107 at a first and the outlet 112 is located at a second end of the third container 110 opposite the first end.

Also, in various embodiments, the first container 101, second container 102, and/or third container are each cylindrical and/or are each are symmetrical about an axis, e.g., an axis of symmetry. In some aspects, the axis of the first container is parallel with the axis of the second container and/or the axis of the third container. In some versions, the axis of the second container is parallel with the axis of the third container.

In some versions of the subject devices, a container, such as the first, second and/or third container includes a solution, such as a preparation solution, which can be a liquid solution. Such a solution can include a buffer solution and/or a lysing agent, such as a cell lysing agent, such as a lysis buffer. According to various aspects, a preparation solution, such as a nucleic acid amplification preparation solution, includes one or more lysing agent, such as one or more detergent. Such a lysing agent can, for example, include detergents, e.g., Tween, Triton X-100, SDS, dichlorodiphenyltrichloroethane (DDT), dithiothreitol (DTT), chaotropic salts, acids and/or bases, pH buffers, beads, solvents, or any combinations thereof. Such an agent can lyse cells of a biological sample to release nucleic acids therefrom. A preparation solution, such as a nucleic acid amplification preparation solution, can also include H2O and/or one or more buffer. Such a solution can be stored within a container, such as a second container of a device while the second plunger is retained within the second container at the first end of the container, wherein the container has a first end coinciding with the first direction 105 and a second end opposite the first direction coinciding with the second direction 106.

In some versions, a device 100 includes an inlet conduit 113 operatively coupled to a valve 107, or a portion thereof, e.g., a fourth opening 205. The inlet conduit can be operatively coupled to the valve at a first end of the conduit and can include an inlet 114, e.g., a sample inlet, at a second end opposite the first end. The inlet conduit 113 can be configured to allow a fluid, such as a biological sample, to flow therethrough into the device from the inlet. In various embodiments, the first container 101 is fluidically connected to the inlet conduit 113 when the valve 107 is in the first configuration and/or not fluidically connected to the inlet conduit 113 when the valve 107 is in the second configuration.

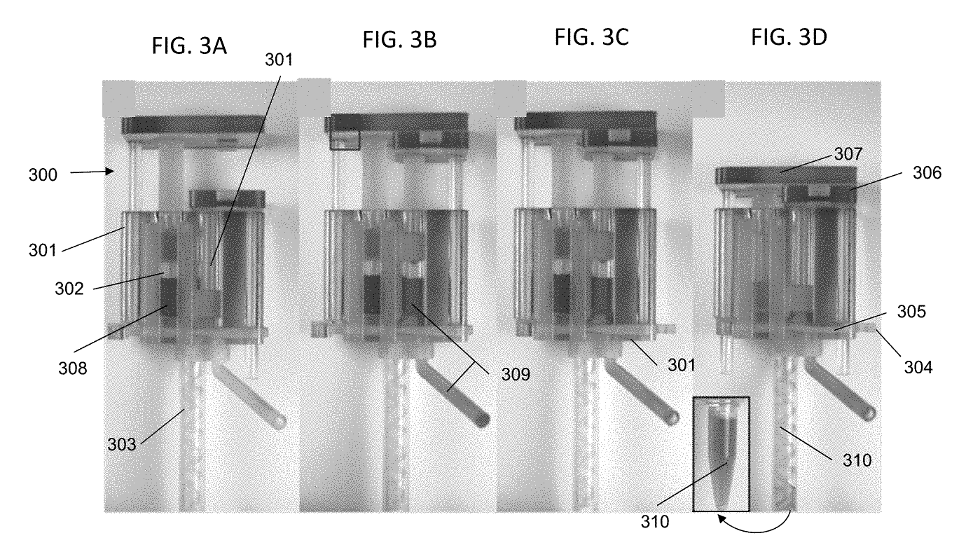

A device 300 can also include a housing 301, as is shown, for example, in FIG. 3. Such a housing is omitted from FIGS. 1 and 2 for clarity. A housing can include the first 301 and/or second 302 and/or third 303 containers and can be integral with any one or combination of the first, second and/or third containers. Here, by “integral” is meant composed of a single piece of integrated material or materials. A housing can contain therein, such as entirely contain between two opposite portions thereof, the first, second and/or third containers, or any combination thereof. A housing can also be fixedly coupled to a first and/or second container. Also, a first and/or second plunger can be actuated with respect to the housing, the first, second and/or third container.

Also, a housing can include a valve receptacle 305 including one or more valve-receiving grooves in which the valve 304 can slide between, for example, a first configuration as shown in FIGS. 3A and 3B and a second configuration, as shown in FIGS. 3C and 3D.

In some embodiments, a first and/or second and/or third container can be cylindrical, and can have a consistent circular, oblong, rectangular, triangular cross-sectional shape and/or diameter and/or circumference along its length. A first and/or second container can extend along a length, such as a length from a first end of the device to a second end of a device opposite the first end, wherein the second end includes the valve. A first and/or second and/or third container can also be integral with a housing of a device and as such, can have edges defined by the edge of the housing.

A first, and/or second and/or third container can have a length ranging from 1 cm to 100 cm, such as from 1 cm to 50 cm, such as from 1 cm to 25 cm, such as from 5 cm to 15 cm, or 100 cm or less, such as 50 cm or less, such as 25 cm or less, such as 15 cm or less, such as 10 cm or less. Also, a first and/or second and/or third container can be cylindrical and can have a cross-sectional diameter ranging from 1 mm to 10 cm, such as 1 mm to 5 cm, such as 1 mm to 1 cm, such as 1 mm to 5 mm, or from 5 mm to 5 cm, such as 5 mm to 3 cm, such as 5 mm to 1 cm. A first and/or second and/or third container can have a cross-sectional diameter of 10 cm or less, such as 5 cm or less, such as 1 cm or less, such as 5 mm or less, or of 15 mm or less, 12 mm or less, or 10 mm, 9 mm, 8 mm, 7 mm, 6 mm, or 5 mm or less. A first and/or second and/or third container can also define an interior volume and/or be configured to receive a sample volume and/or gas, e.g., air, volume therein ranging from 1 mm³ to 3500000 cm³, such as from 1 mm³ to 1000000 cm³, such as from 1 mm³ to 1000 cm³, such as from 1 mm³ to 100 cm³, such as from 1 mm³ to 10 cm³, such as from 1 mm³ to 1 cm³, or from 1 cm³ to 100 cm³, such as from 1 cm³ to 10 cm³, such as from 5 cm³ to 10 cm³.

Furthermore, a first container can have a cross-sectional diameter which is equal to or larger than the cross-sectional diameter of the second and/or the third container. Also, a second container can have a cross-sectional diameter which is equal to or larger than the cross-sectional diameter of the first and/or the third container. Also, a third container can have a cross-sectional diameter which is equal to or larger than the cross-sectional diameter of the first and/or the second container. A ratio of a cross-sectional diameter of a first container to that of a second container or a cross-sectional diameter of a second container to that of a first container can be 2:1, 2.2:1, or 1:1, or 5 or less:1, or 3 or less:1, or 2.5 or less: 1.

In addition, a first plunging element can have a cross-sectional diameter that is equal to or larger than the cross-sectional diameter of the second plunging element. Also, a second plunging element can have a cross-sectional diameter that is equal to or larger than the cross-sectional diameter of the first plunging element. A ratio of a cross-sectional diameter of a first plunging element to that of a second plunging element or a cross-sectional diameter of a second plunging element to that of a first plunging element can be 2:1, 2.2:1, or 1:1, or 5 or less:1, or 3 or less:1 or 2.5 or less:1.

In some variations of the devices, a first container defines a first volume therein and a second container defines a second volume therein. In some versions, the second volume is larger or smaller than the first volume. In some aspects, the ratio of the second volume to the first volume is or the first volume to the second volume is 5 or less:1, such as 3 or less:1, such as 2.5 or less: 1. In some aspects, the ratio of the second volume to the first volume is 2:1, or substantially 2:1, or 2.2:1, or substantially 2.2:1, or 1:2, or substantially 1:2, or 1:1, or substantially 1:1. Also, a biological sample can have a first volume and/or a preparation solution can have a second volume, wherein the ratio of the second volume to the first volume is 2:1, or substantially 2:1, or 2.2:1, or substantially 2.2:1, or 1:2, or substantially 1:2, or 1:1, or substantially 1:1. In some aspects, the ratio of the second volume to the first volume is or the first volume to the second volume is 5 or less:1, such as 3 or less:1, such as 2.5 or less:1. Furthermore, actuating a first plunger and a second plunger in concerted motion can include flowing a volume of solution out of the first and second containers, wherein the volume of solution is composed of a volume of preparation solution and a volume of biological sample, wherein the ratio of preparation solution to biological sample is 2:1, or substantially 2:1, or 2.2:1, or substantially 2.2:1, or 1:2, or substantially 1:2, or 1:1, or substantially 1:1.

In some variations of the devices, a first plunging element defines a first volume and a second plunging element defines a second volume. In some versions, the second volume is larger or smaller than the first volume. In some aspects, the ratio of the second volume to the first volume is 2:1, or substantially 2:1, or 2.2:1, or substantially 2.2:1, or 1:2, or substantially 1:2, or 1:1, or substantially 1:1. In some aspects, the ratio of the second volume to the first volume is or the first volume to the second volume is 5 or less:1, such as 3 or less:1, such as 2.5 or less:1.

In various embodiments, the subject devices can include a first and/or second plunger. Each of the first and/or second plunger can have a plunging element, a handle and/or a locking element. A plunging element, a handle and/or a locking element of a first and/or second plunger can all be operatively connected, integral and/or operatively connectable such that they move in concerted motion. Moving in concerted motion refers to moving together in unison at the same speed at the same time in the same direction in response to a force exerted thereon. For example, a first plunger can move in concerted motion with a second plunger in a direction, such as direction 106, when a force is exerted on the second plunger when a user directly contacts the second plunger but not the first plunger and the second plunger, in turn, exerts force on the first plunger to move both of the plungers together.

A first plunger can include a first plunging element 114 configured to be received within, e.g., entirely within, the first container. A handle of a first plunger 116, or a portion thereof, such as a first and/or second surface, can also be operatively coupled to the first plunging element. Also, a second plunger can include a second plunging element 115 configured to be received within, e.g., entirely within, the second container. A handle of a second plunger 117, or a portion thereof, such as a first and/or second surface, can also be operatively coupled to the second plunging element.

A first and/or second plunger or a portion thereof, e.g., a first plunging element and/or a second plunging element, and/or a first locking element and/or a second locking element, can be cylindrical and can have a length ranging from 1 cm to 100 cm, such as from 1 cm to 50 cm, such as from 1 cm to 25 cm, such as from 5 cm to 15 cm, or 100 cm or less, such as 50 cm or less, such as 25 cm or less, such as 15 cm or less, such as 10 cm or less. Also, a first and/or second plunger or a portion thereof, e.g., a first plunging element and/or a second plunging element, and/or a first locking element and/or a second locking element, can have a cross-sectional diameter ranging from 1 mm to 10 cm, such as 1 mm to 5 cm, such as 1 mm to 1 cm, such as 1 mm to 5 mm, or from 5 mm to 5 cm, such as 5 mm to 3 cm, such as 5 mm to 1 cm. A first and/or second plunger or a portion thereof, e.g., a first plunging element and/or a second plunging element, and/or a first locking element and/or a second locking element, can have a cross-sectional diameter of 10 cm or less, such as 5 cm or less, such as 1 cm or less, such as 5 mm or less, or of 15 mm or less, 12 mm or less, or 10 mm, 9 mm, 8 mm, 7 mm, 6 mm, or 5 mm or less. A first and/or second plunger or a portion thereof, e.g., a first plunging element and/or a second plunging element, and/or a first locking element and/or a second locking element, can also define an interior volume ranging from 1 mm³ to 3500000 cm³, such as from 1 mm³ to 1000000 cm³, such as from 1 mm³ to 1000 cm³, such as from 1 mm³ to 100 cm³, such as from 1 mm³ to 10 cm³, such as from 1 mm³ to 1 cm³, or from 1 cm³ to 100 cm³, such as from 1 cm³ to 10 cm³, such as from 5 cm³ to 10 cm³.

Also, in some aspects, a handle of a first plunger 116, is actuable in the second direction 106 toward the valve 107 when the valve 102 is in the second configuration and/or the first configuration. In some aspects, a handle of a second plunger 117, is actuable in the second direction 106 toward the valve 107 when the valve 102 is in the second configuration but not the first configuration. In some aspects, a handle of a first plunger 116 and/or a handle of a second plunger 117, are concertedly actuable in the second direction 106 toward the valve 107 when the valve 102 is in the second configuration but not the first configuration.

The first and/or second plunger can also each respectively include a handle operatively coupled to a plunging element at a first end of the plunging element opposite a second end of the plunging element, wherein the second end of the plunging element is configured to be received and/or retained within a container. A handle of a first plunger 116, or a portion thereof, such as a first and/or second surface, can also be operatively coupled to a first end of a first locking element 108 opposite a second end of a locking element including a surface for contacting a valve portion to prevent actuation of the plunger. Also, a handle of a second plunger 117, or a portion thereof, such as a first and/or second surface, can also be operatively coupled to a first end of a second locking element 109 opposite a second end of a locking element including a surface for contacting a valve portion to prevent actuation of the plunger. A handle of a first plunger can be configured to be retained between the handle of the second plunger and the valve throughout device operation, such as throughout actuation of the first and and/or second plungers. As such, a portion, e.g., an entire portion, of a handle of a first plunger can be retained, e.g., entirely retained, between at least a portion of a handle of a second plunger and at least a portion of a valve while the first and/or second plunger actuates and/or the valve actuates.

In FIGS. 2A-E, the rectangular blocks at the bottom of each panel provide a top-down view of the valve. Black circles and rings indicate openings in the valve. Slashed circles indicate the presence of a feature that is blocked by the valve. Light circles inside dark circles indicate the presence of a locking element or an open channel for the flow of a solution, e.g., a biological sample, and/or a preparation solution.

A handle of a first and/or second plunger can be a planar plate, such as a rectangular plate, having a thickness extending from a first surface to a second surface opposite the first surface. A handle of a first and/or second plunger can have a length, and/or a width and/or a thickness ranging from 1 mm to 100 cm, such as from 1 cm to 50 cm, such as from 1 cm to 25 cm, such as from 5 cm to 15 cm, or 100 cm or less, such as 50 cm or less, such as 25 cm or less, such as 15 cm or less, such as 10 cm or less, such as 5 cm or less, such as 1 cm or less, such as 0.5 cm or less, such as 1 mm or less. Also, a handle can be substantially rectangular, square, circular, oblong, triangular, or any combination thereof along a cross-section of the handle, such as a cross-section perpendicular to direction 105 and/or direction 106. Also, a first surface and/or a second surface of a handle, such as a first and/or second handle can be perpendicular to direction 105 and/or direction 106.

A handle can also be configured for manual operation, such as for manual actuation of a plunger. Manual operation can include applying force, such as by applying pressure to a surface of a handle, e.g., a first surface and/or a second surface of a handle. Applying pressure, in various aspects of the subject disclosure, includes applying a pressure in an amount which is normally exertable, e.g., tactilely exertable and/or manually exertable, by a human, such a normal or average adult human.

A handle of a second plunger can be configured to receive therein a portion of the handle of the first plunger. Also, a handle of a first plunger can be configured to receive therein a portion of the handle of the second plunger. A handle of a second plunger can include a mating element configured to operatively couple to, such as by mating with, such as by extending within, a receptacle of a handle of a second plunger when the handle of the first plunger contacts the handle of the second plunger.

Also, a first and/or second locking element can have the same shape as a plunging element, e.g., a cylinder, or a different shape. In some embodiments, a first and/or second locking element is cylindrical and has a smaller cross-sectional diameter along its length than that of the first and/or second plunging element. A first and/or second locking element can be a rod, e.g., a solid and/or hollow, rod extending from a handle to a blocking surface configured to contact a valve and thereby prevent a first and/or second plunger from actuating while the valve is in the first and/or second valve configuration. Also, in various embodiments a first and/or second locking element extends along a length from a first end to a second end. A length of a first locking element can be longer or shorter than a length of a second locking element. A length of a first locking element can also be longer or shorter than a length of a first and/or second plunging element. Additionally, length of a second locking element can be longer or shorter than a length of a first and/or second plunging element.

In some versions, the first container 101 and/or the second container includes one or more stopper. A stopper, e.g., 120, can be at an end of a container and include an opening through which a plunging element, such as a second plunging element 115 can protrude while it actuates in the container. The plunger can be operatively, e.g., sealably, connected with the plunging element such that fluid, e.g., air, and/or liquid, e.g., preparation solution, in the container does not move through the seal when the plunging element actuates in the container.

Each of the components of the subject devices, such as the first container, second container, first plunger, second plunger, first plunging element, second plunging element, first handle, second handle, valve, housing, third container, mixing element, first locking element and/or the second locking element, can be composed of a variety a materials, such as a single material, or a plurality of materials, such as two, three, four, five, or ten or more materials. Each of such components can include one or more flexible materials, such as a layer of flexible material coating a core composed of one or more rigid materials. By “flexible,” as used herein is meant pliable or capable of being bent or flexed repeatedly (e.g., bent or flexed with a force exerted by a human hand or other body part) without damage (e.g., physical deterioration). Such components can also include one or more polymeric materials (e.g., materials having one or more polymers including, for example, plastic and/or rubber and/or foam) and/or metallic materials. Such materials can have characteristics of flexibility and/or high strength (e.g., able to withstand significant force, such as a force exerted on it by use, without breaking and/or resistant to wear) and/or high fatigue resistance (e.g., able to retain its physical properties for long periods of time regardless of the amount of use or environment).

According to the subject embodiments, the components of the subject devices, can each be composed of a variety of materials and can be composed of the same or different materials. Materials of interest that any of the device components described herein can be composed of include, but are not limited to: polymeric materials, e.g., photopolymer materials such as Veroclear, and TangoPlus, and/or plastics, such as polytetrafluoroethene or polytetrafluoroethylene (PFTE), including expanded polytetrafluoroethylene (e-PFTE), polyester (Dacron™), nylon, polypropylene, polyethylene, high-density polyethylene (HDPE), polyurethane, etc., metals and metal alloys, e.g., titanium, chromium, stainless steel, etc., and the like. The materials can be transparent or semi-transparent such that a device user can observe a biological sample and/or a preparation solution throughout device operation, such as during mixing. By utilizing translucent materials, fluids are visible as they are transported among chambers of the device, providing visual feedback during operation.

The materials can also be materials that are effectively printed, such as by melting and dispensing in an ordered manner, using a 3D printer. For example, all parts can be designed according to the subject embodiments using 3D CAD software (Solidworks) and fabricated using an Objet 260 multi-material 3D printer (Stratasys, Eden Prairie, Minn., USA).

Materials employed can include one or more semi-transparent photopolymer materials that are a rigid plastic, such as Veroclear and/or TangoPlus and/or poly(methyl methacrylate) (PMMA). Materials employed can also include one or more soft, elastomeric material, such as rubber. Such elastomeric materials can be flexible yet biased to remain in their initial shape when force is exerted thereon.

In various embodiments, all of the components are composed of a strong rigid material such as Veroclear. In some aspects, the first and/or second plunging elements, plungers, stoppers, and/or valve are composed of a strong rigid material such as Veroclear and a second elastomeric material, such as TangoPlus, forming a layer over the Veroclear. The second elastomeric material effectively provided seals in the device, such as seals in the device at sliding surfaces, to prevent liquid seepage therefrom. The second elastomeric material can form a layer over a rigid material having a uniform thickness on a component ranging, for example, from 0.001 to 10 mm, such as from 0.001 to 5 mm, such as from 0.01 to 3 mm, such as from 0.1 to 1 mm. METHODS

Methods for biological sample preparation using the subject devices are included herein. Methods of manufacturing such devices, such as by using 3D printing, are also provided.

In some versions, the methods include preparing biological sample to, for example, produce a prepared biological assay sample. Aspects of the methods can include exposing a biological sample to a preparation solution, e.g., a cell lysing agent and/or a buffer, within a portion of the device to produce a prepared biological assay sample. Producing the prepared biological sample can include exposing, such as by mixing in a third container, a preparation solution to one or more aspects of the biological sample, wherein such exposure results in a change in the biological sample, e.g., cell lysing, such that the modified biological sample or a portion thereof, e.g., nucleic acids, can be further processed and/or analyzed, such as amplified.

In some embodiments of the subject disclosure, a prepared biological assay sample is a biological assay sample that has been processed by exposing the sample to a preparation solution, as described above. Such exposure can prepare the sample for further analysis and can include lysing cells of the sample with a lysing agent of the preparation solution and/or extracting nucleic acids therefrom. Such extracted nucleic acids can be released into a resulting prepared sample solution. In some embodiments, the methods include a step of extracting genomic deoxyribonucleic acid (DNA) from a biological sample. In some versions, the preparation solution is a nucleic acid amplification preparation solution and exposure to the solution prepares nucleic acids of the sample for amplification. After such exposure, the sample is a prepared nucleic acid amplification sample.

According to the subject embodiments, and as illustrated, for example in FIGS. 2B and 2C, the methods include advancing, such as advancing in a first direction 105, a first plunger 103 of a biological assay sample preparation device 100 within a first container 101 of the device to move, such as by flowing, a biological sample into the first container. In such a step, a portion of the first plunger 103, such as a handle 116 can be moved in a direction away from a valve 107 of the device. In such a step, a second plunger 104 is not moved with respect to the valve 107. Also, in such a step, a fluid, such as a liquid, such as a liquid including a biological sample is flowed into the first container through an opening in the valve and via an inlet conduit 113 operably, e.g., fluidically, connected to the first container 101 when the valve is in a first configuration.

Also, advancing a first plunger 103 of a biological assay sample preparation device 100 within a first container 101 of the device to move, such as by flowing, a biological sample into the first container can include moving the first plunger from a first configuration, as shown, for example, in FIG. 2B to a second configuration, as shown in FIG. 2C. In the second configuration, for example, no portion of the first plunger extends into an opening in the valve 107 and as such, the valve can be actuated. In the first configuration, however, a first locking portion 108 extends into an opening in the valve 107 and prevents the valve from being actuated.

Advancing, such as advancing in a first direction 105, a first plunger 103 of a biological assay sample preparation device 100 within a first container 101 of the device to move a biological sample into the first container can include manually advancing the plunger 103 by directly contacting and exerting force on the plunger in a direction away from the valve. The advancing can also be performed automatically, such as by a mechanical and/or electrical component controlled by an electrical control system, such as a central processing unit, exerting force on the plunger. As such, the advancing can include providing a central processing unit with an input including instructions to advance the plunger.

Also, as noted above, in some versions, the first container 101, second container 102, and/or third container are each cylindrical and/or are each are symmetrical about an axis, e.g., an axis of symmetry. In such aspects, advancing, such as advancing in a first direction 105 and/or a second direction 106, a first plunger 103 and/or a second plunger 104 of a biological assay sample preparation device can include moving a first plunging element 114 and/or a second plunging element 115 along the axis of its respective container, such as in the first direction and/or the second direction.

According to the subject embodiments, and as illustrated, for example in FIGS. 2C and 2D, the methods include actuating, such as actuating, such as actuating by sliding, in a first direction 201, a valve 107 of the device from a first configuration as shown, for example, in FIG. 2C, to a second configuration as shown, for example, in FIG. 2D. Such an actuation can include fluidically connecting the first container with a second container of the device including a liquid agent. Such an actuation can also include fluidically connecting the first container and/or the second container of the device with a third container 111 of the device. Such an actuation can also include fluidically disconnecting the first container with the inlet conduit.

Actuating, such as actuating a valve 107 of the device can include manually actuating the valve by directly contacting and exerting force on the valve in a direction perpendicular to an axis defined by a first and/or second and/or third container and/or first and/or second plunger, e.g., direction 201. The actuating can also be performed automatically, such as by a mechanical and/or electrical component controlled by an electrical control system, such as a central processing unit, exerting force on the valve. As such, the actuating can include providing a central processing unit with an input including instructions to actuate the valve.

Also, actuating, such as actuating a valve 107 of the device, can include actuating the valve a distance in a direction, wherein the distance is 1 mm or more, 2 mm or more, 3 mm or more, 4 mm or more, 5 mm or more, 6 mm or more, 7 mm or more, 8 mm or more, 9 mm or more, 10 mm or more, 2 cm or more, 5 cm or more, or 10 cm or more, or 1 mm or less, 2 mm or less, 3 mm or less, 4 mm or less, 5 mm or less, 6 mm or less, 7 mm or less, 8 mm or less, 9 mm or less, 10 mm or less, 2 cm or less, 5 cm or less, or 10 cm or less. Actuating a valve 107 of the device, can include actuating the valve a distance ranging from 1 mm to 10 cm, such as 1 mm to 1 cm, such as 1 mm to 5 mm, each inclusive. As used herein, “inclusive” refers to a provided range including each of the listed numbers. Unless noted otherwise herein, all provided ranges are inclusive.

Embodiments of the methods also include advancing the first plunger 103 in the first container 10l and the second plunger 104 in the second container 102 of the device in concerted motion, such as concerted motion toward the valve. In some versions, advancing the first and/or second plunger in concerted motion can include directly contacting, such as contacting with a body part of a user, such as a hand or a portion thereof, such as a finger, and exerting force on a second handle 117 of a second plunger 104 but not a first handle 116 of a first plunger 113. Such advancement can include exerting force on the first handle 116 from, such as only from, the second handle 116. Such advancement can also include advancing the first handle 116 and the second handle 117 in concerted motion in a direction 106 toward the valve and/or third container. Such a direction can also be parallel with an axis defined by a cylindrical element such as a first container, second container, and/or third container and/or a first plunger and/or second plunger.

In some aspects, advancing the first plunger and the second plunger in concerted motion prepares the biological sample by mixing a biological sample and preparation solution. Such mixing can include providing a turbulent fluid flow by moving the biological sample and/or preparation solution over a mixing element within a third container. Also, advancing the first plunger and the second plunger in concerted motion can include lysing cells of the biological sample.

Advancing the first plunger and the second plunger in concerted motion can also include propelling contents of the first and/or second container into a third container. In some versions, advancing the first plunger and the second plunger in concerted motion can include moving, e.g., flowing, a biological sample out of the first container and into a third container and/or flowing a preparation solution out of the second container and into the third container. In some versions, advancing the first plunger and the second plunger in concerted motion can include moving, e.g., flowing, a biological sample, such as a prepared biological sample and/or a preparation solution out of a third container via an outlet thereof.

Also, advancing the first plunger 103 in the first container 101 and the second plunger 104 in the second container 102 of the device in concerted motion, such as concerted motion toward the valve, can include manually advancing the plungers by directly contacting and exerting force on a plunger, e.g., a second plunger 104, in a direction toward a valve and/or third container. The advancing can also be performed automatically, such as by a mechanical and/or electrical component controlled by an electrical control system, such as a central processing unit, exerting force on one or more plungers. As such, the advancing can include providing a central processing unit with an input including instructions to advance the one or more plungers.

Also, advancing a first plunger 103 within a first container 101 and/or second plunger 104 within the second container 104 in concerted motion, to for example, push fluid into the third container 111, can include moving the first plunger from the second configuration as shown, for example, in FIG. 2D to the first configuration as shown, for example, in FIG. 2E. Such advancement can also include moving the second plunger 104 from an initial configuration, as shown, for example, in FIG. 2D to a subsequent configuration as shown, for example, in FIG. 2E.

When the first plunger is in the second configuration as shown, for example, in FIG. 2D, no portion of the first plunger, such as a first locking element, extends into the valve. Also, when the second plunger is in the initial configuration as shown, for example, in FIG. 2D, no portion of the second plunger, such as a second locking element, extends into the valve. However, when the first plunger is in the first configuration as shown, for example, in FIG. 2E, the first locking element, extends into and through an opening in the valve. Also, when the second plunger is in the subsequent configuration as shown, for example, in FIG. 2E, the second locking element, extends and through an opening in the valve.

Also, in various embodiments of the methods, a preparation solution, such as a lysis buffer is pre-loaded into the second container and/or stored in the second container prior to device operation for mixing. Also in various embodiments, the topmost position of the second plunger is pre-determined by the stopper because the stopper contacts the plunger and prevents it from advancing past a particular distance in a direction, e.g., direction 105.

Furthermore, as is shown, for example in FIG. 3, in some aspects of using a device 300, in an initial step, the user pulls up on the first plunger 306 until it contacts and is stopped by the second plunger 307 or a portion thereof, such as by a second handle thereof. In some versions, a preparation solution 308 is pre-loaded into the second container 302 of the device. Such an action aspirates a biological sample 309 into the first container and simultaneously removes the first locking rod from the valve. The user then slides the valve, opening a path for the second locking element to move therethrough, opening the first container and second container outlets to the third container, closing off the inlet conduit from the first container, and providing a new opening for the first locking element to pass through. In the next step, a user pushes down on the second plunger, and only on the second plunger, e.g., not on the first plunger, wherein the pushing ejects the biological sample and preparation agent through into and/or through the third container 303, wherein the solutions are well mixed to form a mixed solution 310, such as a prepared biological sample, before finally being ejected from an outlet of the third container. The mixed solution 310 is shown in a vial after being ejected from the outlet of the third container in the bottom panel of FIG. 3D.

In the FIG. 3 demonstration, which shows the device 300 assembly and operation, 1150 μL 0.05% (v/v) Sky blue Ateco dye (August Thomson Corp., Glencove, N.Y., USA) was preloaded into the second container and 0.1% Lemon yellow Ateco dye was manually loaded into the first container. These two dye solutions were run through the device 300 and combined to form a green mixed solution, e.g., 310.

In some versions, the methods include building and validating a meter-mix device. The methods of using the device include accurately metering and lysing biological samples, such as human urine samples, for use, for example, in downstream nucleic acid amplification. In some versions, a plurality, e.g., two, plungers and a valve, e.g., a multivalve, generate and control fluid flow through the device.

Device operation according to the subject methods can include three steps that are performed sequentially, and provide rapid, such as 20 sec or less, such as 10 sec or less, such as 5 sec or less, or from 1 to 20 sec or 1 to 10 sec or 5 to 10 sec, and accurate metering and/or mixing. In some versions, the methods of measuring and/or metering samples or other substances do not include manual pipetting or vortexing.

The subject devices and methods can also be used to prepare a sample for use in a devices such as those disclosed in PCT/US2015/000243, U.S. Provisional Application No. 62/096,131, filed Dec. 23, 2014, and/or U.S. Provisional Application No. 62/135,041, filed Mar. 18, 2015, each of which are hereby incorporated by reference in their entirety. As such, the samples, reagents and other aspects of the devices described in the listed applications, can be used in accordance with the subject devices and methods. The subject device can also be applied to deliver a sample, e.g., a prepared sample, to a device as disclosed in the listed applications. Accordingly, the subject device can be configured to operatively connect to such a device and to provide a fluid flow thereto via the connection.

According to the subject methods, to operate a meter-mix device, the user can perform steps including: 1. inserting urine suction tube into a biological sample source, e.g., patient sample, and pulling first plunger, 2. removing biological sample from the biological sample source and slide valve, and 3. pushing second plunger to eject a mixed solution, e.g., a mixed solution including preparation solution and biological sample. The user of the device cannot accidentally perform these operations out of order due to the presence of locking elements attached to the plunger handles. As is illustrated, for example, in FIG. 1A, in the initial position, the first locking element blocks the sliding of the valve, and the valve blocks the movement of the second plunger. When the user pulls up on the first plunger, biological sample is aspirated through the inlet conduit, through the valve, and into the first container. As is illustrated, for example, in FIGS. 1B and 1C, pulling up on the first plunger also releases the first locking element that was blocking the valve. The user then slides the valve, which can be a multivalve, which disconnects the inlet conduit from the first container while providing two new outlets to a third container, one of which is a biological sample outlet and the other for preparation solution that has been pre-stored in the second container. By pre-storing the preparation solution, e.g., lysis buffer, many manual pipetting steps are eliminated and user error is reduced. (16). As is shown in FIG. 1C, sliding of the valve also provides openings for the first locking element and the second locking element. As is shown in FIG. 1D, in another step, a user pushes down on the second plunger, which also pushes the first plunger, ejecting both biological sample and preparation solution through the third container.

### Methods of Manufacturing Devices

According to some versions of the methods, 3D printing is employed to design and prototype the subject devices, which can be macrofluidic devices. Such a device is amenable to diagnostics in limited-resource settings, where speed, accuracy and user-friendly design are important components. In some aspects, multi-material 3D printing technology is employed, which allows composites, e.g., composites of two or more materials, with rigid properties and elastomeric properties to be printed as a single part. In some aspects, the methods include employing multi-material 3D printing to create leak-free seals, such as leak-free seals along sliding-part connections, in the subject devices.

Designing and prototyping leak-proof connections

As noted above, in some embodiments, the methods include performing multi-material 3D printing to produce one or more components of the devices. In multi-material printing, two or more different materials are combined into a single printed part. In some versions of the embodiments, the devices or portions thereof compress one or more fluids, such as liquids and/or gasses while containing the liquids and/or gasses within, e.g., substantially within, one or more containers of the device by preventing leaking. As such, according to the embodiments, multi-material printed parts are employed to generate sealed fluid cavities. The printed parts can include a valve, e.g., multivalve, and/or one or more plungers or portions thereof, e.g., first and/or second plunging elements, applied within the meter-mix devices.

In some aspects, one or more, such as all of the seals, such as interfaces where one device component meets and contacts and/or slides against another, on the device are hermetically sealed. Providing a hermetic seal can be achieved according to the subject methods using Multi jet 3D printing to generate materials jointly composed of two or more materials such as a hard plastic, e.g., Veroclear, and a soft flexible, elastomeric and/or rubber-like material, e.g., TangoPlus. In such embodiments, the components can be composed of a core of a hard, inelastic, and/or inflexible material encapsulated by a layer of a flexible and/or elastomeric material. Multi-material printing can be applied according to the subject methods for fabricating both plungers and/or the valve. An aspect of creating leak-proof connections can include determining the appropriate dimensions, overlap, and the ratio of soft:hard, e.g., inflexible:flexible, and/or inelastomeric:elastomeric, materials to create a strong leak-proof connection in which it is still easy to move the components manually.

For the first and/or second container, a forming fit can include applying a 5 mm to 10 mm, such as an 8 mm diameter container and/or a 5 mm to 10 mm, such as an 8 mm diameter plunging element. In some versions, a diameter of a plunging element core, such as a cross-sectional diameter of a hard and/or solid and/or inelastomeric material, e.g., Veroclear, ranges from 5 mm to 10 mm and can be 7.2 mm. Such an inner core can be surrounded by a layer ranging from 0.1 mm to 1 mm, such as 0.4 mm (5%), in thickness of a soft and/or flexible and/or elastomeric material, e.g., TangoPlus. Also, for the first and/or second container, forming a fit can include applying a 10 mm to 15 mm, such as an 11.31 mm diameter container and/or a 10 mm to 15 mm, such as an 11.31 mm diameter plunging element. Such an element can include a by a layer of flexible and/or elastomeric material, e.g., TangoPlus, having a thickness of 5% or less or 5% or more of the thickness of the inner core of the component. Components of the devices, e.g., valves, and/or first and/or second plunger or portions thereof, e.g., first and/or second plunging elements, can include a solid core, such as a solid core having a cross-sectional length or diameter, encapsulated or surrounded by a layer of flexible and/or elastomeric material having a thickness which is 25% or less, 20% or less, 15% or less, such as 10% or less, such as 5% or less, such as 3% or less, such as 1% or less, or 15% or more, 10% or more, 5% or more, 3% or more, 1% or more, of the cross-sectional length or diameter of the component. Using these parameters can provide hermetically sealed connections capable of generating and holding a vacuum.

At points of contact between device components, e.g., a container and a plunger and/or a plunger and a valve, there can be an overlap of material, e.g., flexible and/or elastomeric material with another flexible and/or elastomeric material or a solid inflexible material, to provide a fluid-tight seal. Such an overlap can range, for example, from 0.01 mm to 1 mm, such as from 0.1 mm to 0.5 mm, such as from 0.2 mm to 0.5 mm, or can be 1 mm or less, such as 0.5 mm or less, such as 0.3 mm or less, such as 0.2 mm or less in thickness.

A valve, or a portion thereof, such as an inner core and/or an outer layer and can have a length, and/or a width and/or a thickness ranging from 1 mm to 100 cm, such as from 1 cm to 50 cm, such as from 1 cm to 25 cm, such as from 5 cm to 15 cm, or 100 cm or less, such as 50 cm or less, such as 25 cm or less, such as 15 cm or less, such as 10 cm or less, such as 5 cm or less, such as 1 cm or less, such as 0.5 cm or less, such as 1 mm or less. In some versions, a valve or a portion thereof, e.g., an inner core, can have a thickness of 1 cm or less, such as 5 mm or less, such as 3 mm or less, such as 2.7 mm or less. In some versions, a valve or a portion thereof, e.g., an outer layer, can have a thickness of 5 mm or less, such as 1 mm or less, such as 0.54 mm or less, such as 0.5 mm or less. Components of the devices, e.g., valves, can include a solid core, such as a solid core having a cross-sectional length and/or width and/or thickness, encapsulated or surrounded by a layer of flexible and/or elastomeric material having a thickness which is 25% or less, such as 20% or less, such as 15% or less, such as 10% or less, such as 5% or less, such as 3% or less, such as 1% or less, or 25% or more, such as 20% or more, 15% or more, 10% or more, 5% or more, 3% or more, 1% or more, of the length, width and/or thickness of the component.

The dimensions of the containers in the housing can be selected to provide the desired air volumes and mixing ratios. To generate the valve seal, an open cavity can be provided through the side of the housing, with raised ridges around each hole for the inlets and outlets. The valve can be 2.7 mm thick, with 0.54 mm TangoPlus (20%) layered on the top and 0.54 mm on the bottom. At the points of contact between the valve and the inlet/outlet ridges, there can be a 0.2 mm overlap where the ridge pushes into the TangoPlus layer (by 3D CAD design). Furthermore, to assist sealing and sliding, silicone oil can be applied to lubricate all contact points at movable interfaces, e.g., interfaces between plunger heads, containers, and/or the valve.

### Kits

The embodiments disclosed herein also include kits including the subject devices and which can be used according to the subject methods. The subject kits can include two or more, e.g., a plurality, three or less, four or less, five or less, ten or less, or fifteen or less, or fifteen or more, biological sample preparation devices or components thereof, according to any of the embodiments described herein, or any combinations thereof.

The kits can include one or more solutions and/or reagents, such as any of those described herein, e.g., preparation solutions and/or biological samples and/or buffers, which can be stored in the kits in containers and/or separate from the devices. In addition, the kits can include any device or other element which can facilitate the operation of any aspect of the kits. For example, a kit can include one or more devices for receiving and/or analyzing one or more characteristics of a sample, e.g., a prepared sample. Kits can also include packaging, e.g., packaging for shipping the devices without breaking.

In certain embodiments, the kits which are disclosed herein include instructions, such as instructions for using devices. The instructions for using devices are, in some aspects, recorded on a suitable recording medium. For example, the instructions can be printed on a substrate, such as paper or plastic, etc. As such, the instructions can be present in the kits as a package insert, in the labeling of the container of the kit or components thereof (i.e., associated with the packaging or subpackaging etc.). In other embodiments, the instructions are present as an electronic storage data file present on a suitable computer readable storage medium, e.g., Portable Flash drive, CD-ROM, diskette, etc. The instructions can take any form, including complete instructions for how to use the devices or as a website address with which instructions posted on the world wide web can be accessed.

### Utility

The sample-to-device interface for diagnostics is an important aspect of nucleic acid amplification testing (NAAT) in LRS. (5,6). The devices and associated methods described herein help optimize the sample-to-device interface. Many NAAT technologies are not amenable to LRS, because NAAT is an intrinsically multistep process involving sample metering, lysis, nucleic acid (NA) purification, amplification, and detection. (7). To be useful in clinical practice in POC or LRS, the entire NAAT workflow should be fully automated, user-friendly (without training or pipetting steps to meet CLIA-waiver), rapid, equipment-free, sensitive, and specific. To equip a portable device with complete sample-in to answer-out functionality requires the appropriate consideration of all upstream and downstream processes.

While many efforts have been taken to automate nucleic acid (NA) purification and amplification, sample metering must always be addressed because a user in LRS or at the POC cannot be asked to pipette accurately. Furthermore, combining sample transfer with the step in which the sample is mixed with the lysis buffer is attractive, because it has the advantage of minimizing the cost and complexity of an integrated diagnostic device, and could benefit such devices being developed in research labs. (8-11). Precise metering is especially critical in NAAT testing of sexually transmitted diseases (STDs), as discussed above.

Currently, there is no standardized way to deliver a known amount of sample mixed with lysis buffer to an LRS- or POC-compatible NAAT diagnostic device. A method for doing so is subject to the following constraints: (i) meter a precise volume of urine with <10% coefficient of variation (CV), (ii) mix urine with premeasured, preloaded lysis buffer at a specific ratio (as determined by the extraction chemistry), (iii) transfer the lysed urine without dripping potentially infectious solution, (iv) perform these operations quickly, in a user-friendly, equipment-free manner that minimizes potential user errors, and (v) maintain the sensitivity and specificity of the overall assay (no loss of nucleic acids to 3D printed surfaces, contamination, or leachates). As described herein, the subject devices and methods overcome such restraints.

In some versions of the methods, multi-material 3D printing is applied for the design and prototyping of an interlock meter-mix device that meters and/or lyses biological samples, e.g., human urine samples, for a workflow compatible with diagnostic testing in limited-resource settings (LRS) and at the point of care (POC). 3D printing includes a set of additive manufacturing techniques that allows the formation of complex 3D structures with minimal restrictions. The emerging technological capabilities of 3D printing bring exciting advancements in the fabrication of micro- and macrofluidic devices, enabling architectures that would be difficult with conventional fabrication techniques such as soft lithography. (1,2). For example, 3D printing has the ability to rapidly prototype and iterate new designs, without needing to tool expensive molds. (3). 3D printing also reduces the design and prototyping time from weeks and months down to hours and days, making prototyping more cost-effective and therefore more accessible—particularly for research labs where needs can change frequently. Because 3D printing is semi-automated, it minimizes assembly time, the requirements for labor, and reproducibility issues, therefore reducing many of the barriers that currently prevent some research labs from prototyping complex 3D parts. (2). The customizable design files generated in computer-aided design (CAD) software can be easily modified in coordination with experiments. Materials used for 3D printing also exhibit a wide range of properties, with varying levels of rigidity, surface roughness, optical clarity, and biocompatibility to fit a diverse range of device requirements. (4). In combination, all of these aspects make 3D printing effective for prototyping fluidic devices relevant to lab-on-a-chip and diagnostics fields.

Furthermore, although there are some limitations to 3D printing capabilities (e.g. dimension limitations related to support material used in printing), the advantages of customizability, modularity and rapid prototyping illustrate the utility of 3D multi-material printing to improve preanalytic sample handling in diagnostics according to the methods and associated devices described herein.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

As is described in greater detail below, Bretherton's prediction was used and tested; using the Bond number to guide designs prevents potentially biohazardous samples from leaking from the device. To validate the meter-mix device with clinically relevant samples, urine spiked with inactivated Chlamydia trachomatis and Neisseria gonorrhoeae was used. A downstream nucleic acid amplification by quantitative PCR (qPCR) confirmed there was no statistically significant difference between samples metered and mixed using the standard protocol and those prepared with the meter-mix device, showing the 3D-printed device could accurately meter, mix and dispense a biological sample, e.g., a human urine sample, with full lysis and without loss of nucleic acids.

### I. Plunger System and Accurate Metering

To accurately meter a fluid, such as a liquid, such as a biological sample, such as urine, the subject plunger device with predetermined start and stop positions can be employed. During device operation, the first plunger, e.g., urine plunger, is pulled up until it contacts the underside of the second plunger, e.g., the lysis buffer plunger. The volume displaced by the plunger was calculated in CAD software, providing an estimate for the volume of urine aspirated into the device. To precisely calibrate metering, the working design was iterated by testing prototypes of the device by aspirating deionized water, weighing the device, and modifying the height of the plunger stoppers to adjust the volume displaced by the plunger.

With diagnostic devices, minimizing dead volumes can help avoid wasting reagents, losing sample, or introducing a source of variability. One strength of 3D printing is that potential sources of dead volume can be identified and reduced during the design process. In designing the subject device four potential sources of dead-volume were identified: urine lost in the suction tube, urine lost in the urine chamber, lysis buffer lost in the lysis buffer chamber, and mixed solution remaining in the static mixer. While a biological sample, such as patient urine can be abundant, and as such, it can be acceptable for the meter-mix device to overfill urine, the final volume of urine ejected from the device is consistent between runs. To ensure accurate, consistent ejected volumes, the dead-volume of the urine suction tube was taken into account while modifying the positions of the plunger stoppers. It should be noted that dead-volume can be reduced by changing the design of the suction tube as required. For the meter-mix device, one consideration was to prevent dead volumes of urine remaining in the urine chamber and the static mixer, which could contribute to differences in the volumes of urine ejected between runs. In particular, a user who sees liquids trapped in the static mixer can be inclined to shake the meter-mix device, introducing error which affects the accuracy of downstream quantitative processes. To remove this dead volume, a pocket of air that sits about the lysis buffer within the lysis buffer chamber is provided according to the subject methods. After urine is aspirated into the device, the heights of the pockets of air are substantially equal (the air initially residing in the suction tube is incorporated into the device during the aspiration step). These two pockets of air produce a blow-out volume of air which removes the dead volumes of urine and lysis buffer that would otherwise remain in the chambers and static mixers.

Table 1 provides data obtained in testing Bretherton's prediction using 3D printed tubes, e.g., containers, of varying diameter.

TABLE 1

| Fluid | Diameter (mm) | Bo | Observed Behavior |
|---|---|---|---|
| Water | 2 | 0.136 | No drip |
| | 2.5 | 0.212 | No drip |
| | 3 | 0.306 | No drip |
| | 3.5 | 0.416 | No drip |
| | 4 | 0.544 | Bubble sticks |
| | 4.5 | 0.688 | Bubble sticks |
| | 5 | 0.850 | Bubble sticks/bubble rises |
| | 5.5 | 1.028 | Bubbles rises |
| Ethanol | 2 | 0.345 | Bubble sticks |
| | 2.5 | 0.539 | Bubble sticks |
| | 3 | 0.776 | Bubble sticks/bubble rises |
| | 3.5 | 1.056 | Bubble rises |
| | 4 | 1.379 | Bubble rises |
| | 4.5 | 1.746 | Bubble rises |
| | 5 | 2.155 | Liquid spills as air column rises |
| | 5.5 | 2.608 | Liquid spills as air column rises |

In some versions of the devices, after biological sample, e.g., urine, is aspirated into the first container, e.g., urine chamber, urine is unable to leak out through the tip of the inlet conduit, e.g., urine suction tube. Bretherton previously examined this aspect, and found the dimensionless bond number, Bo (which relates gravity to surface tension), to be a guiding parameter. (17). The bond number is related to the density difference between the liquid and air, the diameter of the tube, and the surface tension of the liquid. Bretherton predicted that for a vertical tube that is sealed at one end, a bubble contained within will not rise if Bo<0.842.17. Thus, it was hypothesized that in the meter-mix device, if the bond number is low, and a bubble enters the urine suction tube, the bubble will be immobile, preventing solution from dripping out through the tip of the urine suction tube. Bretherton's prediction suggests that the bond number should be minimized, which can be done simply by reducing the diameter of the 3D-printed urine suction tube. However, the diameter is not reduced to an extent that it generates a high resistance to flow, as this would generate a noticeable delay in the filling time and negatively affect the user experience. Tube diameter is also constrained by the 3D printing methods because as tube diameter decreases, it becomes increasingly difficult to remove the support material and clean inside the tube. For the subject device, testing was performed using >1.5 mm diameter sized suction tubes. At the millimeter scale, there was no noticeable delay between pulling up on the urine plunger and filling of the urine chamber.

The Bretherton prediction was tested using 3D-printed device components. A simple plunger system was designed along with suction tubes of varying diameters. In multi-material 3D printing, the printing of support material can be avoided for some geometries and configurations. Straight suctions tubes were printed in the vertical configuration, which does not print support within the suction tube and therefore does not require support cleaning. While some support can be avoided, one limitation of a multi-material printer is that it often prints support material for the bottom layer in contact with the 3D printer's build plate. When one side of the model is printed in contact with support and the other parts of the model are located on the exterior sides of the device, there can be minor differences between dimensions and surface roughness. For example, it was found that when printing straight tubes upright, the diameter on the side of the tube in contact with the 3D printer's build plate was slightly smaller than the opposite opening. A discrepancy between parts of the model in contact with the build plate and open to air is not an exclusively multi-material 3D printing characteristic, but is common to many types of 3D printers. Care was taken to always use the side of the tube in contact with the build plate for the connection to the body of the plunger system.

To test the prediction, the opposite side of the suction tube was used to aspirate solution into the tube. The suction tube was manually disturbed through tapping the tip in order to introduce bubbles, mimicking a real-world user experience where the user bumps the device. There was general agreement between bond number and the Bretherton prediction (Table 1). Using water, for bond number <=0.416, no bubbles entered the device and no fluid dripped from the tip. For bond numbers between 0.544 and 0.688, a bubble entered the tube releasing some drops, but the bubble did not rise and the liquid-air interface at the tip regained stability. Close to the Bretherton prediction at Bo=0.850, bubbles entered the tube and both rise and no rise of the bubble were observed, which seemed to depend on the size of the bubble incorporated. Finally, for a large bond number (1.028), drops were released when the bubble initially entered the tube, the liquid-air interface at the tip regained stability, and bubble rise was shown as predicted by Bretherton. The experiment was repeated using ethanol with similar results. It was also observed that for very large bond numbers (Bo>1=2.155), once the ethanol-air interface at the tip was disturbed, a column of air entered the suction tube, spilling all of the solution out of the tip. Accounting for Bretherton's prediction, the limitations of cleaning support material, and accounting for the pocket of air for blow-out, a suction tube diameter of 2.3 mm was applied in the final design. The surface tension of urine from healthy patients ranges from 48-70 mN/m.18 Using the low value of surface tension at 48 mN/m, a density of 1.01, and a 2.3 mm diameter gives a Bo=0.272.

### II. Accurate Dispensing

The flow rate of each solution, e.g., the biological sample and/or the preparation solution, is determined by the design of the device containers, plungers, inlets and/or outlets. Each container, which is also referred to herein as a chamber, of the device was designed to undergo the same driving pressures over the entire dispensing operation. This can be accomplished by matching the solution height, air pocket height, and plunger heights in both chambers. For example, a 2:1 volume ratio can be obtained by making the area of one chamber twice the area of the second chamber. The cross-sectional area of the channels and outlet valves should also be maintained at the 2:1 ratio to obtain the flow resistance and corresponding volumetric flow rate. The subject device was designed with a 2:1 volume ratio between lysis buffer and urine, but the potential for flow irregularities near the beginning and end of the flow regime was also recognized. If slight inaccuracies during filling cause urine to enter the static mixer prematurely or after all of the lysis buffer has gone through, this could leave some urine unmixed and unlysed. This could lead to inaccuracies during downstream quantification and unlysed bacteria are a biohazard. To address these concerns, the lysis buffer compartment was slightly overfilled leading to a final lysis buffer to urine volume ratio of 2.2:1.

The dispensing accuracy of the device was evaluated using water, green dye, spectrophometer measurements, and a balance. To examine inter-device variability, three different device prototypes were tested, each run in triplicate (Table 2). There was no significant difference among devices for aspiration volume (P=0.46) or the volume expelled (P=0.44). Sample aspiration was found to accurately meter ˜790 μL (<1% CV). As previously described, the blow-out volume of air is responsible for ejecting the final volumes of urine and lysis buffer remaining in the chambers and the static mixer. It was found that pushing the plunger down over the course of 1-2 s led to relatively little error in the final ejection volume (<2% CV). However, pushing the plunger down faster (in <1 s) pushed bubbles through the static mixer and greater volumes of liquid remained in the device, resulting in reduced ejection volumes (˜1350 In real-world applications, it is important to minimize differences resulting from user operation. Future designs can address the issue of plunger speed affecting dead volume by reducing the diameter of the outlets to prevent bubbles from escaping before the fluid. The ratio of solution ejected from the lysis buffer chamber and the urine chamber was calculated by measuring the absorbance of the final ejected solution and comparing it to the green dye loaded into the lysis buffer chamber. Dispensed volumes out of the lysis buffer chamber and urine chamber were similar, with percent deviations of 2.5% and 6.6%.

### III. Static Mixer Design and Mixing Evaluation

A third container and/or mixing element is also referred to herein as a static mixer. To simplify user experience and eliminate mixing by pipetting or vortexing, an on-device Kenics static mixer (KMS) was designed. (19). The flow rates of urine and lysis buffer to exit the outlets at a consistent flow rate had previously been designed. It was predicted that a KMS mixer placed after the lysis buffer and urine outlets would be an efficient way to mix the two streams. The static mixer is composed of alternating left- and right-hand 180° helical twists with 90° offsets between elements. This immobile structure encased within a tube guides the flow of solutions from the center of the tube to the wall of the tube and from the wall to the center. Each element splits and recombines streams of flow, rapidly homogenizing the fluid, similar to mixing by chaotic advection in moving plugs. (14, 20, 21). A KMS static mixer composed of eight elements was designed, with a diameter of 5 mm, and a length:diameter ratio of 1.25:1. Limited by the requirements of removing support material from 3D-printed parts, it was not feasible to print the entire mixer and tube enclosure as a single unit. Instead, a modular approach was used, printing the mixer elements and the mixer case as separate pieces. Both parts were printed in the upright configuration.

In various embodiments, when static mixer elements were printed with the glossy finish setting, only the topmost element was glossy and had different surface roughness and dimensions than the other elements (remaining parts had the matte finish because they were printed in contact with supporting material). To address this issue, the static mixer elements were printed with the matte finish (FIG. 4A). The static mixer elements and the static mixer case were cleaned separately and assembled carefully because the static mixer elements were very prone to breaking (FIGS. 4B-D).

Table 2 provides data obtained in evaluating dispensing accuracy using green dye in water loaded into the lysis buffer chamber and water loaded into the urine chamber.

TABLE 2

| | | | | Calc. | |
|---|---|---|---|---|---|
| | | Aspiration | Ejection | Volume from | Calc. Volume |
| | | Volume | Volume | Lysis Chamber | from Urine |
| Device | Trial | (μL) | (μL) | (μL) | Chamber (μL) |
| 1 | 1 | 782 | 1591 | 1067 | 524 |
| | 2 | 784 | 1613 | 1121 | 492 |
| | 3 | 798 | 1660 | 1135 | 525 |
| 2 | 1 | 796 | 1619 | 1150 | 469 |
| | 2 | 799 | 1630 | 1065 | 565 |
| | 3 | 791 | 1577 | 1120 | 457 |
| 3 | 1 | 788 | 1611 | 1134 | 477 |
| | 2 | 787 | 1586 | 1106 | 480 |
| | 3 | 799 | 1572 | 1099 | 473 |
| AVG | | 791.6 | 1606.6 | 1110.7 | 495.9 |
| STD | | 6.3 | 26.6 | 27.9 | 33.0 |
| CV | | 0.8% | 1.7% | 2.5% | 6.6% |

To evaluate mixing quality, a starch iodine-thiosulfate decolorization was used. The decolorization reaction is an effective method to evaluate mixing because any pockets of unmixed regions will be visible.22 The initial decolorization reaction occurs quickly in a 1:1 iodine:thiosulfate ratio, although a secondary reaction leads to the reappearance of color so higher ratios of iodine:thiosulfate (e.g. 1:1.2 or 1:1.4) can be used. (23-25). For the meter-mix device, a 1:1.05 ratio was used because the design enables rapid mixing within the timescale of the device operation. The starch iodine solution was loaded into the urine chamber through the suction tube, and the sodium thiosulfate was pre-loaded into the lysis buffer chamber. The device mixed the two solutions within the first three to four elements (FIG. 4G). As a control, to confirm that the loss of color is due to mixing and not an artifact of the chemical or optical properties of the 3D printed part, it was also shown the static mixer element fully filled and while mixing with a solution that does not cause decolorization. The meter-mix device was run with starch iodine indicator loaded into both chambers (FIG. 4E) and in a separate experiment with starch iodine loaded into the urine chamber and water loaded into the lysis buffer chamber (FIG. 4F).

FIG. 4 illustrates, for example, a third container 401 and/or mixing element 402 at various stages of use according to the subject methods. The third container and/or mixing element is also referred to herein as a static mixer. For example, FIG. 4 shows assembly of the static mixer (A-D) and an evaluation of mixing quality (E-G). FIG. 4A illustrates freshly printed static mixer elements before cleaning. FIG. 4V illustrates a static mixer element after a 15-min cleaning step. FIG. 4C illustrates a static mixer case. Furthermore, FIG. 4D illustrates an assembled static mixer after inserting a mixing element into the case. In addition, FIG. 4E illustrates an iodine and starch indicator showing flow through the static mixer. FIG. 4F illustrates an iodine and starch indicator mixing with water to demonstrate dilution. Also FIG. 4G illustrates an iodine-thiosulfate de-colorization reaction demonstrating rapid mixing within the first few elements of the mixer.

### IV. Function and Biocompatibility

The subject device was evaluated for compatibility with a routine nucleic acid extraction kit by comparing the metering and mixing steps performed by the device with standard approaches for metering and mixing, e.g., manual pipetting and vortexing. Two concerns are the potential for nucleic acids to bind to 3D printed surfaces, and the potential for compounds from 3D printed materials to leach into the solutions, both of which can negatively affect downstream analysis of nucleic acids. The device was pre-loaded with 1150 μL lysis buffer and aspirated urine spiked with 104 cells/mL of either C. trachomatis (CT) or N. gonorrhoeae (NG) through the suction tube. The multivalve was slid and the plungers were pushed manually, ejecting the solutions through the static mixer and into a 2 mL polypropylene tube. An off-device sample was tested in parallel, with 1100 μL lysis buffer and 500 μL spiked urine (see Table 2) metered by a pipettor and the solution mixed by vortex. No-template controls containing clean urine were also run for both on and off-device conditions.

After mixing, all samples were processed in parallel according to the manufacturer's instructions using the QIAamp Viral RNA Mini kit (recommended for purification of bacterial DNA from urine). Following extraction, nucleic acid concentrations were compared using routine quantitative polymerase chain reaction (qPCR) with primers previously evaluated for the detection of C. trachomatis (26) or N. gonorrhoeae. (27). The threshold cycle for vortex and device-mixed samples were not statistically different, indicating that there was no significant loss of nucleic acids and or material leaching that inhibited downstream analysis. No-template negative controls showed no amplification after 35 cycles.

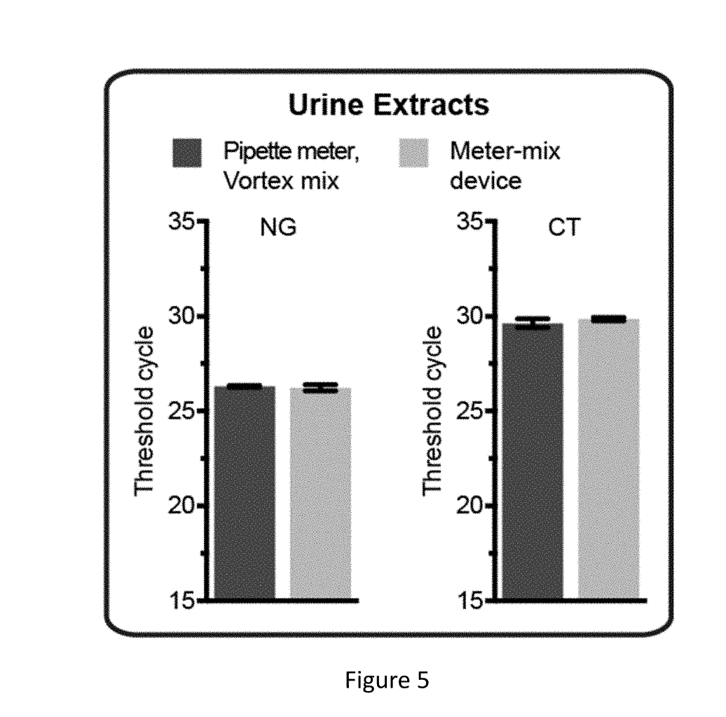

In testing the function and biocompatibility of the tested meter-mix device, urine spiked with inactivated Chlamydia trachomatis or Neisseria gonorrhoeae was metered and mixed with lysis buffer using the meter-mix device. As described above, downstream processing included DNA extraction and qPCR. As is displayed in FIG. 5, results from qPCR were compared to off-device controls utilizing pipetting and vortexing. As noted above, no-template negative controls showed no amplification after 35 cycles.

### V. Meter-Mix Device Cleaning and Assembly

3D-printed parts were cleaned using pipette tips or copper wire and rinsed with water. The urine plunger, lysis buffer plunger, multivalve, and both chambers of the main enclosure chambers were lubricated with viscous silicone oil (Dimethylpolysiloxane 12,500 cSt, Sigma Aldrich, St. Louis, Mo., USA). To assemble, first the urine plunger was inserted into the urine chamber of the main enclosure followed by the lysis buffer plunger into the lysis buffer chamber. The two plunger stoppers were then inserted, locking the topmost position of the lysis buffer plunger. The multivalve was inserted into the main enclosure from the side, and pushed into its final position to preload 1150 uL lysis buffer through the outlet. The multivalve was then moved into its starting position, the urine plunger pushed to the bottom of the chamber, and the urine suction tube and static mixer were attached. For these joints, the outer diameter of the static mixer case (8 mm) and the outer diameter of the urine suction tube (4.5 mm) was sized exactly to the diameter of adapters on the main enclosure. After cleaning, a thin layer of support material remains at the junctions of the main enclosure. Because this support material is shed from the joints during device use, silicone oil was used to enhance the seal.

### VI. Characterization of Metering and Dispensing

In order to evaluate metering and dispensing, the second container, e.g., lysis buffer chamber, was loaded with 1150 μL 0.5% (v/v) green food color dye (The Kroger Co., Cincinnati, Ohio, USA) diluted in deionized water was aspirated into the first container, e.g., urine chamber through the urine suction tube, and mass measured to obtain the aspirated volume (using water density of 1 g/mL). The valve, e.g., multivalve, was pressed and the solution ejected into a pre-tared conical tube to obtain the mass of the solution ejected from the device. The resulting solutions were well-mixed through vortexing. The original 0.5% (v/v) green dye and each resulting solution was diluted by 20×, loaded into a cuvette, and measured with a UV-vis spectrophotometer (Nanodrop 2000c, Thermo Scientific, Wilmington, Del., USA). Measurements were taken at the wavelength where the absorbance was maximal (630 nm), and the ratio was used to determine the volume of solutions ejected from each chamber.

### VII. Iodine-Thiosulfate Decolorization Reaction

Starch indicator, iodine, and sodium thiosulfate solutions were prepared according to the “Handbook of industrial mixing.” (22). In doing so, 1150 μL sodium thiosulfate nonahydrate (0.5 mM, ThermoFisher Scientific, Waltham, Mass., USA) was loaded into the lysis buffer chamber. Starch indicator was prepared by adding 100 mg starch, soluble potato, powder (J. T. Baker, Center Valley, Pa., U.S.) and 20 g potassium iodide to 10 mL deionized water. 50 μL of this starch solution was added to a 1 mL solution of iodine (1 mM, Alfa Aesar, Ward Hill, Mass., USA), coloring the solution dark bluish-purple. The final ratio of iodine:thiosulfate was 0.95:1. A video was taken using the Samsung Galaxy S4 camera, and frames extracted during device operation when the flow fully filled the static mixer (FIGS. 4E-G).

VIII. Extraction and qPCR Experiment

To test device compatibility with biological samples and ensure that downstream nucleic acid analysis was not negatively affected, samples were compared that were metered and mixed on-device against traditional vortex mixing using a commercial nucleic acid extraction kit (QIAamp Viral RNA Mini Kit, 52904). Lysis buffer was loaded with 2 ng/μL carrier DNA (salmon sperm DNA, Thermo Fisher AM9680). Non-infectious CT and NG samples were obtained from ZeptoMetrix Corp. (NATNG-ERCM, NATCT(434)-ERCM, Buffalo, N.Y., USA). Quantitative PCR was performed on a Roche LightCyler 96. PCR reactions consisted of 5 μL SsoFast EvaGreen Supermix (BioRad cat no. 1725200), 2.0 μL of template (extracted spiked urine), 0.5 μL of 20× primer stocks, and 2.5 μL nuclease-free water. The primers used (26,27) were previously evaluated for the detection of either CT or NG. Final primer concentration in the reaction was 500 nM. Thermal cycling consisted of a 3 min initial denaturation step at 95° C., followed by 40 cycles of 20 s at 95° C., 20 s at 62° C., and 20 s at 72° C. Melt analysis was applied to confirm specific product for all reactions.

### IX. Results

It was shown that multi-material 3D printing can be used to produce a meter-mix device that accurately meters biological sample, e.g., urine, and completely mixes it with preparation solution, e.g., lysis buffer, in a format that meets the requirements for a downstream NAAT compatible with LRS and POC settings. The 3D-printed device accurately aspirated predetermined volumes into a urine chamber with a coefficient of variation of 0.8%. Urine and lysis buffer were dispensed through a KMS static mixer at a 2.2:1 mixing ratio. Printing with translucent materials enabled visual confirmation of fluid movement and showed that mixing occurred within the first few elements of the static mixer, with homogenization and lysis later verified by qPCR. Printing with multi-material 3D printer enabled use of a combination of composites to create fluid-tight, e.g., air-tight and water-tight, seals that slide without leaking or losing vacuum pressure. Using a 3D printer also helped address the potential for sample dripping, a biohazardous concern when working with bodily fluids and potentially dangerous solutions, as Bretherton's prediction was successfully tested for bubble rising through several prototype iterations and identify optimal tube dimensions that ensured the sample did not drip.

The subject 3D-printed devices were designed to optimize the user's experience: operation is simple, e.g., three steps; locking elements protect against user error; neither pipetting nor vortexing are required; and the entire device operation can be completed within 5 s or less or 10 s or less. The devices were validated by lysing urine samples spiked with CT/NG and performed downstream processes to quantify nucleic acids through qPCR. The results confirmed that the 3D-printing materials (Veroclear and TangoPlus) were biocompatible; no loss of nucleic acids was observed and devices performed equally well compared with the standard protocol of pipettor metering and vortex mixing in a polypropylene tube. Finally, it was demonstrated that the performance of the meter-mix device matched the performance of standard laboratory protocols for metering and mixing, with a substantially shorter time period for device operation.

The meter-mix device described here can also be applied in a variety of applications such as sequencing, dilutions, and/or chemical syntheses. Because the meter-mix device simplifies and accelerates workflow, protects against user error and provides a user-friendly experience, it can have future application in research labs and limited-resource settings. For example, time-sensitive laboratory measurements can require metering and mixing on the timescale of single digit seconds rather than the tens of seconds required for pipetting.

Throughout the course of device development, the 3D printing workflow was an effective for of prototyping as compared with other methods, such as soft lithography. Prototyping with 3D printing was rapid, and enabled steps including design, test, redesign, and reprint of a prototype in the period of a single day. For small parts that can be printed in less than a few hours, it was possible to iterate multiple designs within in a single day according to the subject methods. The ease with which parts can be modified after having developed the initial design allowed the printing of multiple variations of the meter-mix device at once and as such, allowed the effective determination of the optimal architecture of each part in a single experiment. This was useful for determining the diameter of the suction tube, setting the parameters for the static mixer, and adjusting the fit for the seals. Modularity was also found to be an important advantage with 3D printing. Parts can be built as separate components and later reassembled, reducing build time (which relies heavily on z-axis height). It is also easier to validate and iterate with individual components than to redesign and reprint an entire device.

### REFERENCES

All publications and patents cited in this specification are herein, including those listed below, are incorporated by reference as if each individual publication or patent were specifically and individually indicated to be incorporated by reference and are incorporated herein by reference to disclose and describe the devices, methods and/or materials in connection with which the publications are cited.
1. C. M. B. Ho, S. H. Ng, K. H. H. Li and Y.-J. Yoon, Lab Chip, 2015, 15, 3627-3637.
2. B. C. Gross, J. L. Erkal, S. Y. Lockwood, C. Chen and D. M. Spence, Anal. Chem., 2014, 86, 3240-3253.
3. A. Waldbaur, H. Rapp, K. Lange and B. E. Rapp, Anal. Methods, 2011, 3, 2681-2716.
4. A. Pilipović, P. Raos and M. \check{S}ercer, Int. J. Adv. Manuf. Tech., 2007, 40, 105-115.
5. A. Niemz, T. M. Ferguson and D. S. Boyle, Trends. Biotechnol., 2011, 29, 240-250.
6. P. Craw and W. Balachandran, Lab Chip, 2012, 12, 2469-2486.
7. R. W. Peeling, K. K. Holmes, D. Mabey and A. Ronald, Sex. Transm. Infect., 2006, 82 Suppl 5, v1-6.
8. D. Lee, Y. T. Kim, J. W. Lee, D. H. Kim and T. S. Seo, Biosens. Bioelectron., 2016, 79, 273-279.
9. Q. Tian, Y. Mu, Y. Xu, Q. Song, B. Yu, C. Ma, W. Jin and Q. Jin, Anal. Biochem., 2015, 491, 55-57.
10. R. C. den Dulk, K. A. Schmidt, G. Sabatte, S. Liebana and M. W. Prins, Lab Chip, 2013, 13, 106-118.
11. A. V. Govindarajan, S. Ramachandran, G. D. Vigil, P. Yager and K. F. Bohringer, Lab Chip, 2012, 12, 174-181.
12. W. Huang, C. A. Gaydos, M. R. Barnes, M. Jett-Goheen and D. R. Blake, Sex. Transm. Infect., 2013, 89, 108-114.
13. CDC, Reported STDs in the United States. 2012 National Data for Chlamydia, Gonorrhea and Syphilis, 2014.
14. J. R. Papp, J. Schachter, C. A. Gaydos and B. Van Der Pol, Recommendations for the Laboratory-Based Detection of Chlamydia trachomatis and Neisseria gonorrhoeae 2014, 2014, 63, 1-19.
15. S. Begolo, D. V. Zhukov, D. A. Selck, L. Li and R. F. Ismagilov, Lab Chip, 2014, 14, 4616-4628.
16. S. Makwana, B. Basu, Y. Makasana and A. Dharamsi, Int. J. Pharm. Investig., 2011, 1, 200-206.
17. F. P. Bretherton, J. Fluid. Mech., 1961, 10, 166-188.
18. C. O. Mills, E. Elias, G. H. Martin, M. T. Woo and A. F. Winder, J. Clin. Chem. Clin. Biochem., 1988, 26, 187-194.
19. A. W. Etchells and C. F. Meyer, in Handbook of Industrial Mixing, John Wiley & Sons, Inc., 2004, DOI: 10.1002/0471451452.ch7, pp. 169, 391-477.
20. H. Song, D. L. Chen and R. F. Ismagilov, Angew. Chem., Int. Ed., 2006, 45, 7336-7356.
21. H. Song, M. R. Bringer, J. D. Tice, C. J. Gerdts and R. F. Ismagilov, Appl. Phys. Lett., 2003, 83, 4664-4666.
22. D. A. R. Brown, P. N. Jones, J. C. Middleton, G. Papadopoulos and E. B. Arik, in Handbook of Industrial Mixing, John Wiley & Sons, Inc., 2004, DOI: 10.1002/0471451452.ch4, pp. 145-256.
23. A. D. Awtrey and R. E. Connick, J. Am. Chem. Soc., 1951, 73, 1341-1348.
24. S. Hashimoto, Y. Chikamochi and Y. Inoue, Chem. Eng. Sci., 2012, 80, 30-38.
25. P. J. Carreau, I. Patterson and C. Y. Yap, Can. J. Chem. Eng., 1976, 54, 135-142.
26. J. B. Mahony, K. E. Luinstra, J. W. Sellors and M. A. Chernesky, J. Clin. Microbiol., 1993, 31, 1753-1758.
27. B. S. Ho, W. G. Feng, B. K. Wong and S. I. Egglestone, J. Clin. Pathol., 1992, 45, 439-442.

Although the foregoing invention has been described in some detail by way of illustration and example for purposes of clarity of understanding, it is readily apparent to those of ordinary skill in the art in light of the teachings of this invention that certain changes and modifications can be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A biological assay sample preparation device, the device comprising:
a. a first container and a second container;
b. a first plunger actuable within the first container in a first direction and a second direction opposite the first direction;
c. a second plunger actuable within the second container in the first direction and the second direction; and
d. a valve actuable between a first and second configuration,
wherein the first container is fluidically connected to the second container when the valve is in the second configuration and not fluidically connected to the second container when the valve is in the first configuration, and
wherein the first plunger and the second plunger are concertedly actuable in the second direction toward the valve when the valve is in the second configuration.

2. The device of claim 1, wherein the first plunger comprises a first locking element and the valve comprises a first opening for receiving one or more portion of the first locking element therein in the first configuration.

3. The device of claim 1, wherein the second plunger comprises a second locking element preventing the second plunger from actuating in the first direction when the valve is in the first valve configuration.

4. The device of claim 3, wherein the valve comprises a second opening for receiving one or more portion of the second locking element therein in the second configuration.

5. The device of claim 1, further comprising a third container, wherein the third container is not fluidically connected to the first or second container when the valve is in the first configuration and is fluidically connected to the first and second container when the valve is in the second configuration.

6. The device of claim 5, wherein the third container comprises a mixing element.

7. The device of claim 5, wherein the third container comprises an outlet.

8. The device of claim 1, wherein the second container comprises a preparation solution.

9. The device of claim 1, wherein the device further comprises an inlet conduit, and wherein the first container is fluidically connected to the inlet conduit when the valve is in the first configuration and not fluidically connected to the inlet conduit when the valve is in the second configuration.

10. The device of claim 1, further comprising a housing containing the first and second containers.

11. The device of claim 10, wherein the valve is slidably coupled to the housing and slides within the housing to actuate between the first and second configuration.

12. The device of claim 1, wherein the valve comprises an inner core and an outer layer surrounding the inner core, wherein the inner core comprises a first material and the outer layer comprises a second material different than the first material.

13. The device of claim 1, wherein the first plunger comprises an inner core and an outer layer surrounding the inner core, wherein the inner core comprises a first material and the outer layer comprises a second material different than the first material.

14. The device of claim 1, wherein the second plunger comprises an inner core and an outer layer surrounding the inner core, wherein the inner core comprises a first material and the outer layer comprises a second material different than the first material.

15. A method of preparing a biological sample, the method comprising:
a. advancing a first plunger of a biological assay sample preparation device within a first container of the device to move a biological sample into the first container;
b. actuating a valve of the device from a first configuration to a second configuration and thereby fluidically connecting the first container with a second container of the device comprising a preparation solution; and
c. advancing the first plunger in the first container and the second plunger in the second container of the device in concerted motion toward the valve,
wherein advancing the first plunger and the second plunger in concerted motion prepares the biological sample by mixing the biological sample and the preparation solution.

16. The method of claim 15, wherein the first plunger comprises a first locking element and the valve comprises a first opening for receiving one or more portion of the first locking element therein, and wherein advancing the first plunger within the first container to move the biological sample into the first container comprises removing the first locking element from the first opening.

17. The method of claim 15, wherein the second plunger comprises a second locking element and wherein advancing the first plunger within the first container to move the biological sample into the first container comprises contacting the second locking element with the valve and thereby blocking the second plunger from actuating in the first direction.

18. The method of claim 15, wherein the valve comprises a second opening for receiving one or more portion of the second locking element therein in the second configuration, and wherein advancing the first plunger and the second plunger in concerted motion comprises inserting one or more portion of the second locking element into the second opening.

19. The method of claim 15, wherein advancing the first plunger in the first container and the second plunger in the second container of the device in concerted motion comprises flowing the biological sample and the preparation solution into a third container of the device.

20. A method of manufacturing a biological assay sample preparation device, the method comprising:
a. 3-dimensionally (3D) printing device components comprising:
i. a first container and a second container;
ii. a first plunger actuable within the first container in a first direction and a second direction opposite the first direction;
iii. a second plunger actuable within the second container in the first direction and the second direction; and
iv. a valve actuable between a first and second configuration, and
b. assembling the components to produce a biological assay sample preparation device.
